(19) United States
(12) Patent Application Publication
(10) Pub. No.: US 2003/0139438 A1
(43) Pub. Date: Jul. 24, 2003

(54) Diagnosis of the parkinsonian condition

(76) Inventors: Jonathan Brotchie, Manchester (GB); Michael Hill, Oldham (GB); Alan Crossman, Manchester (GB); Erwan Bezard, Manchester (GB)

Correspondence Address: NIXON & VANDERHYE, PC, 1100 N GLEBE ROAD, 8TH FLOOR, ARLINGTON, VA, 22201-4714, US

(21) Appl. No.: 10/300,914

(22) Filed: Nov. 21, 2002

Related U.S. Application Data
(60) Provisional application No. 60/331,781, filed on Nov. 21, 2001.

(51) Int. Cl. A61K 31/485

(52) U.S. Cl. 514/282

(57) ABSTRACT

The present invention relates to a method of diagnosis of the parkinsonian condition in presymptomatic subjects. Additionally, the invention relates to a method for the differential diagnosis of early Parkinson's disease and differentiating Parkinson's disease patients from patients with other movement disorders.

The present invention relates to a method of diagnosis of the parkinsonian condition in presymptomatic subjects.

Additionally, the invention relates to a method for the differential diagnosis of early Parkinson's disease and differentiating Parkinson's disease patients from patients with other movement disorders. The invention will thus identify patients who will benefit from therapies focussed on either modifying the progress of Parkinson's disease or in providing appropriate symptomatic treatment.

Movement and other disorders due to dysfunction of the basal ganglia and related brain structures are of major socio-economic importance. Such disorders can occur as a consequence of inherited or acquired disease, idiopathic neurodegeneration or they may be iatrogenic. The spectrum of disorders is very diverse, ranging from those associated with poverty of movement (akinesia, hypokinesia, bradykinesia) and hypertonia (e.g. Parkinson's disease, multiple system atrophy (MSA), progressive supranuclear palsy (PSP) and some forms of dystonia) to the involuntary movement disorders (hyperkinesias or dyskinesias e.g. Huntington's disease, levodopa-induced dyskinesia, ballism, some forms of dystonia).

Parkinson's disease and related conditions represents one of the most prevalent diseases associated with poverty of movement. Parkinsonian symptoms manifest as a syndrome of symptoms characterised by slowness of movement (bradykinesia), rigidity and/or tremor. Parkinsonian-like symptoms are seen in a variety of conditions, most commonly in idiopathic parkinsonism (i.e. Parkinson's Disease) but similar symptoms also manifest in disorders such as MSA, PSP, Wilson's disease and essential tremor.

It is widely appreciated that the primary pathology underlying Parkinson's disease is degeneration. in the brain, of the dopaminergic projection from the substantia nigra to the striatum and in particular a reduction in D₂-dopamine receptor mediated neurotransmission. This has led to the widespread use of dopamine-replacing agents (e.g. L-DOPA and apomorphine) as symptomatic treatments for Parkinson's disease and such treatments have been successful in increasing the quality of life of patients suffering from Parkinson's disease.

However, dopamine-replacement treatments do have limitations, especially following long-term treatment. Problems can include a wearing-off of the anti-parkinsonian efficacy of the treatment and the appearance of a range of side-effects which manifest as abnormal movements (dyskinesias), such as chorea and dystonia, which are associated with Di-dopamine receptor stimulation in the striatum. Ultimately, these side-effects severely limit the usefulness of dopaminergic treatments.

Many attempts have been made to develop novel dopamine replacement therapies which will obviate or mitigate these side-effects. However, such attempts have generally met with limited success and there remains a need to develop new and improved ways in which the parkinsonian condition may be treated.

Given the abovementioned difficulties in treating movement disorders it is important that clinicians are able to diagnosis such disorders at an early stage. Early diagnosis enables clinicians to implement a treatment early in the development of the disorder and thereby improve the chances of reversing the development of the disorder or at least delaying the development of the disorder and/or delaying side-effects associated with conventional therapies. Ideally a clinician would be able to diagnose the existence of a disorder, or be able to predict a predisposition to developing a movement disorder, before symptoms of the disorder manifest. Such identification of the disease while it was in the presymptomatic stage would allow the introduction of disease modifying therapies to prevent the appearance of symptoms or slow the progression of the disease to extend the period for which the subject is free of symptoms.

Patients suffering from parkinsonism can present with similar symptoms to patients with disorders such as progressive supranuclear palsy, Wilson's disease, multiple systems atrophy and essential tremor. A positive response to the anti-Parkinson's disease drug L-DOPA is a good diagnostic indicator of the parkinsonian condition (rather than the abovementioned disorders) but has the disadvantage that it can prime patients and cause subsequent problems in treatment (e.g. for development of dyskinetic side effects). Accordingly a non-dopaminergic diagnostic method would be desirable.

It is therefore an object of the present invention to provide a new method of diagnosing the existence of the parkinsonian-like condition, or a predisposition to developing it.

According to a first aspect of the present invention, there is provided a method of diagnosing the existence of a parkinsonian-like condition, or a predisposition to developing such a condition, in a subject comprising:

(i) administering to said subject an agent that blocks effects of metenkephalin; and

(ii) monitoring said subject for the development of, or worsening of, parkinsonian symptoms; wherein the development of said symptoms indicates that said subject has, or is predisposed to developing, said parkinsonian condition.

By “a parkinsonian-like condition” we mean a syndrome of symptoms characterised by slowness of movement (bradykinesia), rigidity and/or tremor. Parkinsonian symptoms are seen in a variety of conditions, most commonly in idiopathic parkinsonism (i.e. Parkinson's Disease) but also following treatment of schizophrenia, exposure to toxins/drugs and head injury.

By “blocks effects of met-enkephalin” we mean that the agent reduces the activity of receptors at which met-enkephalin acts as an agonist. Agents include receptor antagonists as well as other agents such as antibodies raised against the receptor, blockers of receptor signal transduction, agents that increase enkephalin breakdown or agents that decrease synaptic release of enkepahlins.

The inventors have found, to their surprise, that agents which block the effects of met-enkephalin cause the development of parkinsonian symptoms in a subject which is predisposed to develop conditions such as Parkinson's disease or in a subject which is in the early stages of developing such a condition but has not yet developed any symptoms.

Although we do not wish to be bound by any hypothesis, we believe the usefulness of agents that block the effects of met-enkephalin as diagnostic tools may be explained by the pathophysiological actions the inventors believe may be attributed to the enkephalins in the brain.

The principal pathological characteristic of Parkinson's disease (PD) is the progressive death of pigmented dopamine (DA) neurons of the Substantia Nigra pars compacta (SNc). It is thought that parkinsonian signs appear when dopaminergic neuronal death exceeds a critical threshold: 70-80% of striatal nerve terminals and 50-60% of SNc pericarya.

GABAergic efferents from the striatum to the external segment of the pallidal complex (GPe) coexpress enkephalin and are thought to be overactive in the 1-methyl-4-phenyl-1,2,3,6-tetrahydropyridine (MPTP)-treated primate model of PD. This increased activity is generally assumed to play a role in the genesis of parkinsonian symptoms by causing dis-inhibition of the subthalamic nucleus (STN) and, thus, overactivity of basal ganglia outputs from the internal segment of the globus pallidus (GPi) and the substantia nigra pars reticulata (SNr). However. both the role of this cotransmission in the generation of parkinsonian symptoms and the nature of any functional interaction between GABA and enkephalin are not known to the art.

Enkephalin is derived from preproenkephalin (PPE-A) and mRNA levels of PPE-A are good indications of enkephalin levels.

In the light of the above the inventors noted that:

(a) enkephalin biosynthesis is negatively regulated by the dopaminergic tone (e.g. as particularly evidenced in mice lacking the DA D2 receptors);

(b) large striatal dopaminergic depletion or tonic decrease in DA release is thought to be necessary to produce significant changes in striatal preproenkephalin-A (PPE-A) mRNA levels; and

(c) little is known regarding the regulation of PPE-A expression during the progression of movement disorders and more particularly, prior to the emergence of parkinsonian symptoms.

The combination of factors (a), (b) and (c) inspired the inventors to perform experiments (see below) to investigate the role of enkephalins in movement disorders. They established that an up-regulation of striatal PPE-A mRNA levels occurs before the appearance of parkinsonian motor disabilities. Furthermore increased metenkephalin transmission in the external pallidal segment delays the onset of symptoms in parkinsonism. Accordingly the inventors realised that transient blocking of metenkephalin activity in pre-symptomatic subjects allows the symptoms of parkinsonism to manifest. Furthermore, in early Parkinson's disease elevated met-enkephalin will act to limit the severity of symptoms. Accordingly, the inventors realised that transient blocking of met-enkephalin activity in early Parkinson's disease would allow more severe parkinsonian symptoms to become manifest. In disorders, other than Parkinson's disease, such as PSP and MSA, where there are symptoms similar to those seen in the parkinsonian state but no evidence to support a role for metenkephalin in limiting the severity of symptoms, transient blocking of met-enkephalin will not exacerbate symptoms. Thus the invention provides for a method for the differential diagnosis of parkinsonian-like syndromes.

These experiments lead the inventors to the surprising discovery that agents reducing or blocking the actions of met-enkephalin may be administered to a subject and, depending upon the subject's health status, will have different effects. Agents reducing the actions of met-enkephalin administered to normal subjects have no significant effect on the development of parkinsonian symptoms whereas subjects in the early stages of Parkinson's disease, or predisposed to developing it, manifest symptoms of the disorder after treatment with agents reducing the actions of metenkephalin.

Therefore agents which block met-enkephalin activity may be used in a diagnostic test according to the first aspect of the invention to induce symptoms of parkinsonism in subjects predisposed to develop, or in subjects in the early stages, of parkinsonian condition such as Parkinson's disease.

Furthermore the inventors have found that the method according to the first aspect of the invention may be used to distinguish between early Parkinson's disease and a variety of other movement disorders associated with a poverty of movement. Wilson's disease, progressive supranuclear palsy, some forms of dystonia, multiple systems atrophy and essential tremor are all movement disorders associated with a paucity of movement which present with symptoms similar to parkinsonism. However, met-enkephalin treatment does not cause symptoms to become more apparent in subjects in the early stages of such disorders. By contrast subjects in the early stages of a Parkinson's disease do respond to met-enkephalin treatment. The ability to be able to make an early diagnosis of parkinsonism and also to distinguish between different types of movement disorder represents a particular advantage of using the diagnostic method according to the first aspect of the invention.

The method according to the first aspect of the invention is particularly useful when early diagnosis of, or a predisposition to develop, Parkinson's disease is required in human patients. When a clinican recommends somebody for a diagnostic test according to the first aspect of the invention, a human subject should be placed in a clinically controlled environment (an observation ward in a hospital, neurologists office, general practitioners office, or the like) where a clinician can monitor and score (using conventional behavioural indices such as the standard parkinsonian rating scale, the UPDRS) the behaviour of the subject to evaluate whether or not parkinsonian symptoms transiently develop or, if already present, worsen, following administration of the agent. When the test has been completed subjects should be kept under observation until a clinician is satisfied that their behaviour has returned to normal. Patients who do exhibit Parkinsonian symptoms, or in whom symptoms worsen transiently, during the duration of the test may be treated at an early stage to reduce the severity of Parkinson's disease, delay its on-set or even prevent symptoms from developing. For instance, presymptomatic diagnosis may allow neuroprotective strategies to be implemented at an early stage before critical neural damage occurs (e.g. 70-80% of striatal dopaminergic neuronal death or 50-60% of SNC pericarya dopaminergic neuronal death).

A variety of agents may be used according to the present invention to block the effects of met-enkephalin and temporarily reveal/exacerbate parkinsonism symptoms. For instance the agent may be:

(i) an opioid receptor antagonist (e.g. naloxone (a non selective antagonist) or naltrindole (a selective 6-opiold antagonist);

(ii) agents which decrease the synaptic release of met-enkephalin in the GPI/GPe (calcium channel blocking agents, e.g. verapamil, or potassium channel activating agents e.g. diazoxide, levcromakalim); and

(iii) agents that increase the metabolism (i.e. breakdown) of met-enkephalin (e.g. peptidases or small molecule enkephalinase activators) By way of example, naloxone may be employed to assess whether a subject with no parkinsonian symptoms is a case of pre-symptomatic Parkinson's disease. A single injection of naloxone (ideally in the range of 10-100 mg) should be administered by subcutaneous injection. Within one to three minutes, the effects of naloxone will become apparent. The doctor or other healthcare professional assesses the level of parkinsonian symptoms before administration of naloxone and every 5 minutes subsequently. The level of parkinsonism may be assessed by rating Part III of the UPDRS (United Parkinson's Disease Rating scale) i.e. assessment of tremor, rigidity, bradykinesia, balance, speech If the patient is pre-symptomatic Parkinson's disease these symptoms will appear transiently. If symptoms appear they will typically be completely reversible and will subside within 20 minutes of the initial time of naloxone injection. Therefore a clinician should ensure that the subject is supervised for at least 20 minutes after naloxone treatment and thereafter until the clinician is satisfied that the subject has returned to normal.

The inventors further realised that the method according to the first aspect of the invention may be adapted such that it may be used to screen compounds to test whether or not a compound is likely to cause Parkinson's disease. Compounds may be administered to test animals (e.g. rats or primates) for a predetermined length of time following which the test animals may be treated with an agent that blocks metenkephalin activity. Should the animals then develop parkinsonian symptoms it would indicate that the test compounds are linked to causing conditions such as Parkinson's disease. Such a method would have significant advantages over current methods used to assess the potential neurodegenrative properties of novel agents with respect to toxicity to dopamine neurons and thus propensity to induce parkinsonism. Thus a simple behavioural test (e.g administration of naloxone 10 mg/kg subcutaneously) could be applied easily and analysed within minutes and would replace time consuming histological and neurochemical analysis of dopamine cells and other indices of dopamine transmission in animals receiving long term treatment with a drug. Furthermore, unlike post-mortem measures of dopamine loss the test could be applied repeatedly throughout the treatment period and would thus significantly reduce the number of animals needed to assess the propensity of a drug to induce parkinsonism. This method would be especially useful for assessing the toxicity of compounds that are thought to have a propensity to damage the dopamine system with long-term administration e.g. pesticides and herbicides.

According to a second aspect of the present invention there is provided a method of screening a compound to test whether or not said compound causes a parkinsonian-like condition, or a predisposition to developing such a condition, in a subject comprising:

(i) administering to said subject a test compound for a predetermined length of time;

(ii) administering to said subject an agent that blocks effects of metenkephalin; and

(iii) monitoring said subject for the development of, or worsening of, parkinsonian symptoms; wherein the development of said symptoms to a greater extent than seen in control subjects that only receive said agent indicates that said compound causes, or predisposes said subject to developing said parkinsonian-like condition.

It will be appreciated that the screen may be further adapted such that the method is used to test the efficacy of putative medicaments that have neuroprotective properties or anti-parkinsonian properties. In this case the putative medicament may be tested to see if it will prevent the development of parkinsonian symptoms which arise when a compound that is known to cause conditions such as Parkinson's disease is given to a test animal prior to treatment with an agent that blocks met-enkephalin activity.

Therefore according to a third aspect of the invention there is provided a method of screening the efficacy of a putative medicament for neuroprotective properties or anti-parkinsonian properties, in a subject comprising:

(i) administering to said subject said putative medicament and a compound that is known to cause a Parkinsonian-like condition for a predetermined length of time;

(ii) administering to said subject an agent that blocks effects of metenkephalin; and

(iii) monitoring said subject for the development of parkinsonian symptoms; wherein the development of said symptoms to a lesser extent than seen in control subjects that only receive said compound indicates that said putative medicament has neuroprotective properties or anti-parkinsonian properties.

### BRIEF DESCRIPTION OF THE DRAWINGS

The invention will be further illustrated by way of example, with reference to the accompanying drawings, in which:

FIG. 1 illustrates a neurochemical analysis from 2.2 of the Example in which (A) Putamen and (B) caudate nucleus content in DA, DOPAC and HVA are illustrated wherein 100% corresponds to the control values, *=comparison with D0, P<0.05 and +=comparison with D25, P<0.05;

FIG. 2 represents histograms from 2.3 of the Example showing variations in levels of PPE-A mRNA in the striatum (putamen on the left and the caudate nucleus on the right) of monkeys of D6, D12, D15 and D25 groups at the rostral (A), mid (B), and caudal levels (C) wherein DL=dorsolateral region, DM=dorsomedial region, VL=ventrolateral region, VM=ventromedial region, BODY CD=body of the caudate nucleus, *=comparison with D0, P<0.05 and +=comparison with D15 and D25, P<0.05; and

FIG. 3 represents autoradiograms from 2.3 of the Example of coronal sections showing PPE-A mRNRA expression at the caudal level in the caudate nucleus and putamen of D0, D6, D12, D15 and D25 groups.

FIG. 4 shows the effect of the opioid receptor antagonist naltrexone on activity counts in a rodent model of pre-symptomatic Parkinson's disease. ** P<0.01 compared to vehicle; unpaired t-test.

### EXAMPLE

The correlation between met-enkephalin levels and a predisposition to develop a parkinsonian condition was assessed by measuring PPE-A MRNA levels in an animal model. The inventors assessed PPE-A mRNA expression and striatal DA content following a chronic MPTP administration protocol in monkeys that produces a progressive parkinsonian state. Groups ranged from normal to full parkinsonian through asymptomatic lesioned monkeys.

The data presented in this Example lead the inventors to realise that agents which inhibit met-enkephalin activity may be used according to the methods of the invention.

1. Materials and Methods

Animals

Experiments were conducted on twenty five female cynomolgus monkeys (Macaca fascicularis, Shared Animal Health, Beijing, PR of China; mean age=3.1. ±0.3 years; mean weight=2.8 ±0.2 kg). Animals were housed in individual primate cages under controlled conditions of humidity (50±5%0, temperature (24 ±1° C.), and light (12 h light/dark cycles), food and water were available ad libitum and their care supervised by veterinarians skilled in the healthcare and maintenance of nonhuman primates. Experiments were carried out in accordance with European Economic Community (86/6091/EEC) guidelines for care of laboratory animals. The effect of nontreatment on both motor behaviour and PPE-A expression had been previously compared with that of the administration of saline in order to rule out any possible interference. Accordingly MPTP-treated animals were compared with “nontreated” controls in this study. This procedure has been chosen to minimise the number of animals used. In addition, brain tissues acquired for the present experiments are being used for further experiments.

Experimental Protocol

Five monkeys were killed at the beginning of the study and were considered as day 0 (D0) controls. The remaining 20 were treated with daily (9:00 am) injections of MPTP hydrochloride (0.2 mg/kg, i.v.; Sigma, St Louis, Mo.) in saline according to a previously described protocol (Bezard et al., 1997 Neuroscience 81 p 399-404; Bezard et al., 1997 Brain Res. 766 p 107-112; and Bezard et al., 1999 Eur. J. Neurosci. 11 p 2167-2170). This protocol derives a reproducible MPTP cumulative dosing regime that leads to the first appearance of parkinsonian clinical signs after 15±1 injections (3.0±0.02 mg/kg). Five presymptomatic monkeys were killed on day 6 (i.e., after 6 injections; D6 Group), five presymptomatic monkeys at day 12 (i.e. after 12 injections; D12 group), five at day 15 after appearance of overt symptoms (i.e. after 15 injections; D15 group), and the remaining five fully parkinsonian monkeys at day 25 (i.e. after 15 injections; D25). All animals were killed by sodium pentobarbital overdose (150 mg/kg, i.v.) and the brains were removed quickly after death. Each brain was bisected along the midline and the two hemispheres were immediately frozen by immersion in isopentane (−45° C.) and then stored at −80° C. Tissue was sectioned at 20 μm in a cryostat at −17° C., thaw-mounted onto gelatin-subbed slides, dried on a slide warmer and stored at −80° C.

Behavioral Assessment

Animal behavior was assessed daily (2 p.m) on a parkinsonian monkey rating scale (Bezard et al., 1997 Neuroscience 81 p399-404; and Bezard et al., 1997 Neuroreport 8 p435-438) using videotape recordings of monkeys in their cages in addition to clinical neurological evaluation. During each session two examiners evaluated the animals' levels of motor performance, coaxing them to perform various tasks by offering appetizing fruit. A simultaneous independent and blind assessment was made by a third examiner watching a video recording. The minimal disability was 0 and the maximum score was 25. Differences in rating were discussed regularly to eliminate observer idiosyncrasy. Bradykinesia was tested objectively at the beginning of each session by assessing the mean time required to pick up three pieces of fruit positioned 5 cm apart as previously described (Bezard et al., 1997 Neuroscience 81 p399-404). A maximum time of 60 s was allowed to perform the test.

Neurochemical Analysis

The extent of striatal DA denervation was assessed by measuring levels of DA, 3,4-dihydroxyphenylacetic acid (DOPAC) and homovanillic acid (HVA) in both the caudate nucleus and the putamen using high-pressure liquid chromatography with electrochemical detection as previously described (Bezard et al., 1997 Neuroscience 81 p399-404) with minor modifications. After sections have been freeze-dried (−60° C., 40.10⁻³ Atm) for 2 h, the putamen and caudate nucleus regions were separately scrapped off and sonicated in 200 μl of HC10₄ 0.1 N containing 3,4-dihydroxybenzylamine as an internal standard. The homogenates were then centrifugated at 27,000 g for 20 min at 4° C. Pellets were retained for quantification of protein content by the Bradford assay (Bezard et al., 1997 Neuroscience 81 p 399-404). The high pressure liquid chromatography system consisted of a pump (Beckham, Fullerton, Calif.) connected to a stainless steel separation column packed with Hypersil 50DS (Beckman). Electrochemical detection was carried out using a BAS LC-4B detector (Waters Milford, Mass.) with a glassy carbon working electrode, a Ag/AgCl reference electrode and an amperometric detector. Detector potential was set at +0.8V versus the reference electrode. Concentrations of DA and metabolites were calculated using a computing integrator (Gold Nouveau V 1.6, Beckman). Mean and SEM values were calculated for both putamen and caudate nucleus for each group.

Preproenkephalin-A in Situ Hybridization

In situ hybridization histochemistry was performed as previously described by Henry et al. (1999 Exp. Neurol. 155 p204-220) using a ³⁵S-radiolabeled oligonucleotide probe (Gibco BRL) corresponding to amino acids 130-145 of the human sequence of PPE-A. One microliter of the oligonucleotide was tailed by the isotope of 37° C. in a mixture containing 10 μl sterile water, 12.5 μl reaction buffer (sodium cocodylate 120 mM and dithiothreitol 100 mM), and 2 μl terminal deoxynucleotide transferase (all reagents DuPont/NEN) with 7 μl of [³⁵S] ATP (82.5 μCi, NEN). Following 60 min incubation, the labelled probe was purified utilizing Bio-spin chromatography columns (Bio-Rad) centrifuged at 1100 g for 4 min (Z382K, Hermel). Five times volume of eluted [³⁵S]dATP-labeled probe of 1 M dithiothreitol was added. One microliter of this solution was counted by a liquid scintillation counter (Tricarb, 1500 Packard) to asses the efficiency of labeling. Sections were allowed to hybridize at 42° C. for 18 h with 150 μl of hybridization solution (50% formamide, 4× standard sodium citrate (SSC), 10% dextran sulfate, 10 mM dithoithreitol, and labeled-probe up to a final concentration of 3×10⁶ cpm/ml). Stringent washes were carried out for 30 min at room temperature in 1×SSC, 30 min at 55° C. in 1×SSC, and 10 min at 55° C. in 0.1 SSC. Once dehydrated and fully air-dried, both slides and autoradiographic microscale standard (Amersham) were exposed to β-max Hyperfilm (Amersham) for 14 days at 4° C., developed in Kodak D-19 developer and fixed in Kodak Unifix. Control experiments showed that specific hybridization signal was eliminated by unlabeled probe in excess and by pre-treatment of the slides with ribonuclease A (20 μg/ml).

Analysis of in Situ Hybridization Signal

Densitomeric analysis of autoradiographs was performed using an image analysis system (Image Pro Plus, v3.0.01, Media Cybernetics L.P., Atlanta, Ga.) as previously described (Henry et al., supra). The optical density of the autoradiogram was assessed for the striatum at three rostrocaudal levels in accordance with the functional organisation of the striatum: a rostral level, including the caudate, putamen, and nucleus accumbens; a midlevel, including the caudate, putamen, and GPe; and a caudal level including the body of the caudate, the putamen, and both the GPe and GPi. Where appropriate, caudate and putamen were divided into dorsolateral, dorsomedial, ventrolateral, and ventromedial quadrants for analysis. Four sections per animal, per striatal level were analyzed by an examiner blind with regard to experimental condition. Optical densities were averaged for each region in each monkey and converted to amount of radioactivity bound by comparison to the standards. Mean radioactivity bound and SEM were then calculated for each group.

Statistical Analysis

Statistical analysis of the radioactivy bounds was performed using a three-way ANOVA (variables being the striatal level, the striatal region, and the group). A two-way ANOVA was used to estimate overall significance of comparisons of HPLC results (variables being striatal region, i.e., caudate or putamen, and the group). A one-way ANOVA was used for comparison of bradykinesia test recordings. If significant, ANOVAs were followed by post hoc t test comparisons by the method of Bonferroni. Kruskal-Wallis nonparametric test was used to compare parkinsonian scores and, if significant, followed by post hoc t test comparisons by the method of Dunn. All analyses were completed using STATA program (Intercooled stata 6.0, stata corporation, college station, Tex.). A probability level of 5% (P<0.05) was considered significant.

2. Results

2.1 Behavior

Repeated MPTP treatment had a significant effect on both the parkinsonian rating score (KW=23.5; P<0.0001) and the bradykinesia test (F(4.20=184.7; P<0.0001). As normally seen with this administration protocol, monkeys at D6 and D12 were not parkinsonian (parkinsonian score of 0 for all animals at both time points). Furthermore, the mean duration of the bradykinesia test was not significantly different (respectively, 2.4±0.3 and 2.6±0.5s) from D0 monkeys (3.0±0.4s) (P>0.05). These two groups (D6 & D12) were therefore considered as asymptomatic. Monkeys of both the D15 and D25 groups exhibited parkinsonian motor abnormalities (respectively, median 11 (range 10-14) and median 17 (range 15-19); P<0.05 as compared to D0, D6, and D12 groups and P<0.05 between them). The mean duration of the bradykinesia test was significantly increased compared to D0 for the D15 group (19.9±9.1 s; P<0.05), whereas D25 monkeys could not perform the test, reflecting their inability to initiate a voluntary movement (60 s; P<0.05).

2.2 Neurochemical Analysis

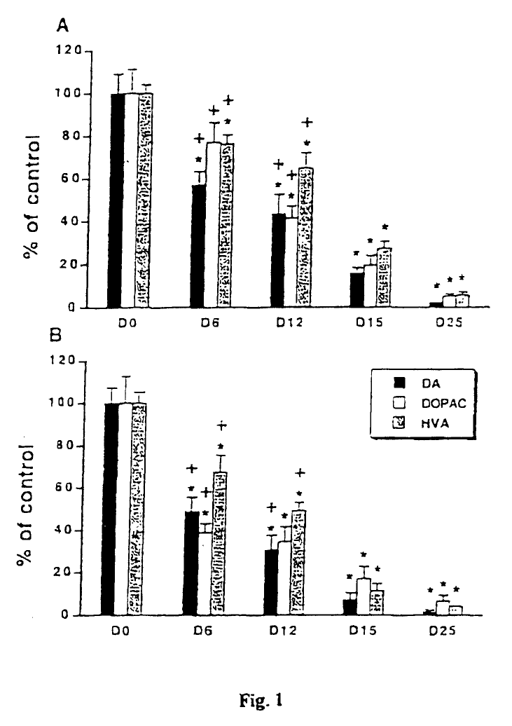

The extent of the dopaminergic lesion was determined by measuring the DA, DOPAC, and HVA levels in the putamen and in the caudate nucleus. MPTP treatment significantly affected the DA and metabolite levels since a group effect was observed (F(4.40=81.7; P<0.0001), whereas there was no significant difference of lesion between putamen and caudate nucleus (F(1.40)=3.1) as well as for the interaction of these two variables (F(4.40)=0.4) (FIG. 1). D0 DA content was 141.5 ±13.2 pg/μg of protein in the putamen and 139.1±10.2 pg/μg of protein in the caudate nucleus. DOPAC content was 16.5±1.9 pg/μg of protein in the putamen and 14.7±1.8 pg/μg of protein in the caudate nucleus. HVA content was 120.5±4.9 pg/μg of protein in the putamen and 130.8±7.0 pg/μg of protein in the caudate nucleus.

When compared to D0 values (control), the level of DA in the putamen was significantly reduced, by 42.7% in the D6 group (t=−60.4, P<0.05). DA levels were dramatically decreased, by 97.9%, in the D25 group (t=−138.6, P<0.05) (FIG. 1A). It should be noted that the depletion of DA reached 56.3% in the D12 group, which was asymptomatic (t=−79.8, P<0.05). Comparable significant decreases were observed for DOPAC and HVA levels in the putamen (FIG. 1A) as well as for all three compounds in the caudate nucleus (FIG. 1B). DA, DOPAC, and HVA levels in the putamen were significantly lower in the D25 group as compared to both D6 (respectively, t=−78.2, P<0.05; t=−11.9, P<0.05 t=−85.5, P<0.05) and D12 groups, i.e., the asymptomatic groups (respectively, t=−58.8, P<0.05; t=−6.0, P<0.05; t=−71.7, P<0.05) (FIG. 1A). The same significant differences were observed in the caudate nucleus (FIG. 1B).

2.3 Preproenkephalin-A in Situ Hybridisation

The three-way ANOVA analysis showed a significant difference for the group (F_{(2,420)}=144.8; P<0.0001), the striatal level (F_{(2,420)}=56.6; P<0.0001), and the striatal region variables (F_{(7,420)}=47.8; P<0.0001) as well as for the interactions between group and striatal level variables (F_{(8,420)}=11.5; P<0.0001), between group and striatal region variables (F_{(28,420)}=6.9; P<0.0001), and finally between all the three variables (F_{(55,420)}=1.4; P<0.05).

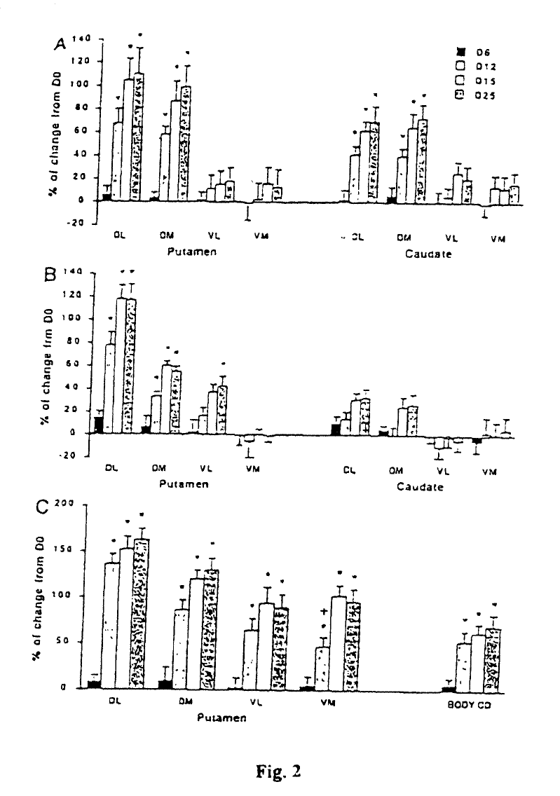

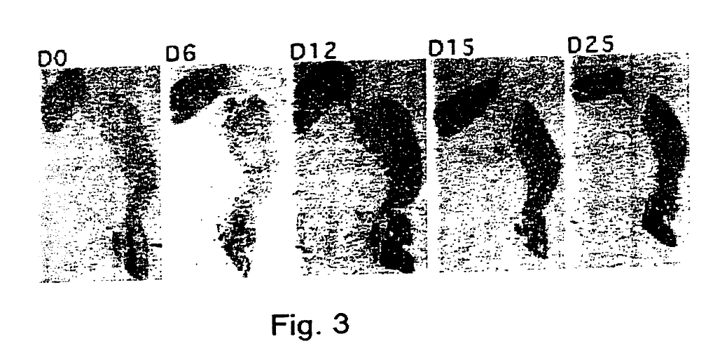

The fully parkinsonian monkey (i.e., D25 group) exhibited pronounced increases in PPE-A mRNA levels in dorsal putamen at the rostal level (t=45.8, P<0.05 in the dorsolateral region; t=39.6, P<0.05 in the dorsomedial region) (FIG. 2A), the midlevel (t=44.8, P<0.05 in the dorsolateral region: t=22.2, P<0.05 in the dorsomedial region) (FIG. 2B), and the caudal level (t=62.4, P<0.05 in the dorsolateral region; t=52.8, P<0.05 in the dorsomedial region) (FIGS. 2C and 3) in comparison with that of group D0. Regarding the ventral region of the putamen, the PPE-A mRNA level was also augmented significantly in both the ventrolateral (t =33.6, P<0.05) and the ventromedial regions (t=35.4, P<0.05 in the ventromedial part) at the caudal level (FIGS. 2C and 3) and in the ventrolateral region of the midlevel (t=16.6, P<0.05) (FIG. 2B). Comparable increases were observed in the D25 group also in the dorsal caudate nucleus at the rostral level (t=29.4, P<0.05 in the dorsolateral region; t=28.8, P<0.05 in the dorsomedial region) (FIG. 2A) and in the body of the caudate at the caudal level (t=27.6, P<0.05) (FIG. 2C). Group D15, which exhibited mild parkinsonian symptoms, showed the same distribution of increased levels of PPE-A MRNA (FIG. 2) except in the ventrolateral region of the putamen at the midlevel, where there was no increase (t=14.6) (FIG. 2B).

There was no significant difference of PPE-A mRNA levels in group D6 compared to D0 group. However the inventors were surprised to find that group D12, although asymptomatic, showed a sharp increase in PPE-A mRNA levels many striatal regions (FIGS. 2 and 3). Group D12 PPE-A mRNA levels were significantly different from group D0 values in the dorsal putamen at the rostral level (t=28.2, P<0.05 in the dorsolateral region; t=23.6, P<0.05 in the dorsomedial region) (FIG. 2A), the midlevel (t=30.2, P<0.05 in the dorsolateral region: t=13.2, P<0.05 in the dorsomedial region) (FIG. 2B), and the caudal level (t=52.4, P<0.05 in the dorsolateral region: t=35.2, P<0.05 in the dorsomedial region) (FIG. 2C), in the ventrolateral region of the caudal putamen (t=24.2, P<0.05) (FIG. 2C), as well as in the dorsal caudate nucleus at the rostral level (t=17.6, P<0.05 in the dorsolateral region; t=17.1, P<0.05 in the dorsomedial region) (FIG. 2A) and in the body of the caudate at the caudal level (t=21.0, P<0.05) (FIG. 2C).

3. Discussion

These data indicate that PPE-A mRNA level is upregulated in MPTP treated, asymptomatic monkeys showing a putaminal DA depletion of 56.3% (D12). This demonstrates that an increase in met-enkephalin expression precedes the development of a parkinsonian condition and lead the inventors to realise that met-enkephalin acts to delay the development of parkinsonian symptoms. Given this realisation, the inventors were able to develop the method according to the fist aspect of the invention whereby agents which block met-enkephalin activity can be used to induce parkinsonian symptoms in presymptomatic subjects and thereby act as a diagnostic tool.

The increase in PPE-A mRNA levels observed in the striatum of parkinsonian monkeys (D15 and D25 groups) is in accordance with earlier reports in DA-depleted animals and humans. A more pronounced increase in PPE-A expression was observed in the dorsolateral and dorsomedial regions of both the caudate nucleus and the putamen, with this increase being most prominent in the putamen as compared with the caudate nucleus, particularly at the midlevel. These regional differences in PPE-A mRNA levels have been previously reported in MPTP-treated monkeys and are thought to reflect differences in dopaminergic innervation. However, the inventors are unaware of any prior art which would suggest that PPE-A expression is increased in asymptomatic/presymptomatic subjects.

### EXAMPLE 2

Reserpine (3 mg/kg, s.c, in 3% glacial acetic acid) was administered to rats to deplete dopamine levels.

After 48 hours the animals had a dopamine depletion but this is insufficient to cause parkinsonian symptoms (i.e the dopamine level was not less than 20% of normal levels). In this situation the animals represent a model of pre-symptomatic subjects, i.e. they have a dopamine depletion but no symptoms.

Following administration of an agent that blocks the actions of metenkephalin, naloxone (10 mg/kg I.p), parkinsonian symptoms became manifest. The parkinsonan symptoms were reversible and were not observed one hour after the naloxone injection where the animals returned to the presymptomatic state. No parkinsonian symtoms were seen in control animals (i.e. animals treated with naloxone, or the vehicle thereof, but not treated with reserpine). This Example demonstrates that parkinsonian symptoms may be induced in presymptomatic subjects/subjects predisposed to develop a parkinsonian condition by following the method according to the present invention.

### EXAMPLE 3

Reserpine was administered to rats to deplete dopamine levels. After 48 hours, the animals have a dopamine depletion but this is insufficient to cause severe parkinsonian symptoms and animals move significantly when they are placed into a novel environment. In this situation the animals represent a model of pre-symptomatic patients, i.e. they have a dopamine depletion but no overt symptoms. Following administration of an agent that blocks the actions of met-enkephalin, naltrexone (10 mg/kg i.p), parkinsonian symptoms appeared and the animals movements were less.

1.1. Methods

1.1.1 Treatments.

Male Sprague-Dawley rats were split into two groups A and B. Rats in both groups received a subcutaneous administration of reserpine (3 mg/kg). The rats were allowed to recover for 48 hours post reserpine treatment at which point they are considered to represent a model of pre-symptomatic Parkinson's disease.

After the 48 hours Group A were treated with vehicle (water) and group B were treated with naltrexone (10 mg/kg) and placed in locomotor monitoring apparatus (see below).

1.1.2 Assessment of Activity.

The locomotion of the rats in Groups A and B following either vehicle or naltrexone treatment was measured over the 5 minute period immediately after drug administration using Benwick locomotor monitors. These locomotion monitors consist of a visually-shielded open-field arena, the perimeter of which is surrounded by a series of infra-red beams arranged at 5 cm intervals. PC-based software (Amlogger) assesses the number of beams broken. The number of beams broken as part of a locomotor movement (activity counts) was measured.

1.2 Results

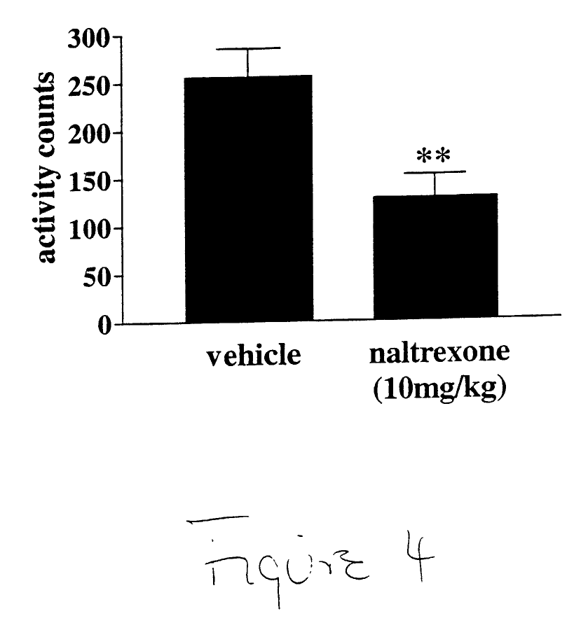

FIG. 4 illustrates that animals in Group A exhibited locomotion when placed in the activity monitoring boxes. This locomotion represents normal exploratory behaviour following introduction into a novel environment, i.e. the activity monitors. However, activity counts were significantly less in the naltrexone-treated group (B) than the vehicle treated group (A). This demonstrates that blocking opioid activity causes the emergence of a parkinsonian state (i.e. less locomotion) in pre-symptomatic animals, suggesting that increased opioid transmission may be a compensatory mechanism to delay the onset of parkinsonian symptoms and that opioids antagonists can make symptoms manifest in situations where they would not normally be seen.

1. A method of diagnosing the existence of a parkinsonian-like condition, or a predisposition to developing such a condition, in a subject comprising:
(iii) administering to said subject an agent that blocks effects of metenkephalin; and
(iv) monitoring said subject for the development of, or worsening of, parkinsonian symptoms; wherein the development of said symptoms indicates that said subject has, or is predisposed to developing, said parkinsonian condition.

2. A method of diagnosis according to claim 1 wherein the subject is a human being.

3. A method of diagnosis according to claim 1 wherein the agent is an opiold receptor antagonist.

4. A method of diagnosis according to claim 3 wherein the agent is naloxone.

5. A method of diagnosis according to claim 4 wherein naloxone is administered to said subject as a single injection of naloxone and said subject is monitored to assess the level of parkinsonian symptoms before administration of naloxone and every 5 minutes subsequently for at least 20 minutes after naloxone treatment.

6. A method of diagnosis according to claim 3 wherein the agent is naltrindole.

7. A method of diagnosis according to claim 1 wherein the agent decreases the synaptic release of met-enkephalin.

8. A method of diagnosis according to claim 1 wherein the agent increases the metabolism of met-enkephalin.

9. A method of screening a compound to test whether or not said compound causes a parkinsonian-like condition, or a predisposition to developing such a condition, in a subject comprising:
(i) administering to said subject said compound for a predetermined length of time;
(ii) administering to said subject an agent that blocks effects of metenkephalin; and
(iii) monitoring said subject for the development of, or worsening of, parkinsonian symptoms; wherein the development of said symptoms to a greater extent than seen in control subjects that only receive said agent indicates that said compound causes, or predisposes said subject to developing said parkinsonian-like condition.

10. A method of screening according to claim 9 wherein the agent is an opioid receptor antagonist.

11. A method of screening according to claim 10 wherein the agent is naloxone.

12. A method of screening according to claim 10 wherein the agent is naltrindole.

13. A method of screening according to claim 9 wherein the agent decreases the synaptic release of met-enkephalin.

14. A method of screening according to claim 9 wherein the agent increases the metabolism of met-enkephalin.

15. A method of screening the efficacy of a putative medicament for neuroprotective properties or anti-parkinsonian properties, in a subject comprising
(i) administering to said subject said putative medicament and a compound that is known to cause a Parkinsonian-like condition for a predetermined length of time;
(ii) administering to said subject an agent that blocks effects of metenkephalin; and
(iii) monitoring said subject for the development of parkinsonian symptoms; wherein the development of said symptoms to a lesser extent than seen in control subjects that only receive said compound indicates that said putative medicament has neuroprotective properties or anti-parkinsonian properties.

16. A method of screening according to claim 15 wherein the agent is an opioid receptor antagonist.

17. A method of screening according to claim 16 wherein the agent is naloxone.

18. A method of screening according to claim 16 wherein the agent is naltrindole.

19. A method of screening according to claim 15 wherein the agent decreases the synaptic release of met-enkephalin.

20. A method of screening according to claim 15 wherein the agent increases the metabolism of met-enkephalin.

21. A method of diagnosis according to claim 7, wherein the agent is a calcium channel blocking agent or a potassium channel activating agent.

22. A method of diagnosis according to claim 21, wherein the agent is selected from the group consisting of verapamil, diazoxide and levcromakalim.

23. A method of diagnosis according to claim 13, wherein the agent is a calcium channel blocking agent or a potassium channel activating agent.

24. A method of diagnosis according to claim 23, wherein the agent is selected from the group consisting of verapamil, diazoxide and levcromakalim.

25. A method of diagnosis according to claim 19, wherein the agent is a calcium channel blocking agent or a potassium channel activating agent.

26. A method of diagnosis according to claim 25, wherein the agent is selected from the group consisting of verapamil, diazoxide and levcromakalim.
